(19) United States
(12) Patent Application Publication
(10) Pub. No.: US 2015/0320987 A1
(43) Pub. Date: Nov. 12, 2015

(54) Pediatric Drain and Tube Management Device

(71) Applicant: Clinton Peter Burns, Rising Sun, MD (US)

(72) Inventor: Clinton Peter Burns, Rising Sun, MD (US)

(21) Appl. No.: 14/273,543

(22) Filed: May 8, 2014

(51) Int. Cl. A61M 27/00; A41B 13/06; A47D 13/08; A61M 25/02

(52) U.S. Cl. CPC A61M 27/00; A61M 25/02; A41B 13/06; A47D 13/08; A61M 2025/0206

(57) ABSTRACT

The present invention is a pediatric drain or tube management device. The device has a unitary swaddling device having a main portion, a first lateral portion adjacent to the main portion, a second lateral portion adjacent to the main portion and opposite the first lateral portion, and a covering portion. Each lateral portion has an aperture extending therethrough and an end having a fastening means affixed thereto. The covering portion is positioned exterior to the lateral portions and has a fastening means affixed thereto. The present unitary swaddling device is configured such that a pediatric patient having a medical tube is placed upon the main portion of the device, the first lateral portion is folded over the patient and the second lateral portion is folded over the first lateral portion, such that the medical tube is extended through the apertures in the lateral portions and the fastening means is used to secure the drain in place, and the covering portion is folded over the lateral portions and the secured drain.

### CROSS-REFERENCE TO RELATED APPLICATIONS

Not Applicable.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

1. Field of the Invention

The present invention relates to the field of medical devices or garments for humans or other animals, and to procedures for using such devices or garments. More particularly, the present invention is directed to pediatric patients or non-pediatric patients of similar body size. The invention more particularly relates to a method and device for protecting, anchoring, and supporting medical drains or devices used in the treatment or maintenance of patients in need of such drains or devices.

2. Description of the Related Art

The present invention relates to medical drain and tube supporting garments and methods of using such garments, as well as analogous methods or devices. In modern medical care, there is often the need to use specialized tubes or drains. These tubes or drains may include tubes for specialized procedures such as gastrostomy or peritoneal dialysis. Similar tubes are used for nutritional support of the patient or for such bodily functions as urine removal. Managing the various tubes or devices in a manner that is effective and comfortable for the patient has become a very important matter to address.

Just as seen in adult patients, pediatric patients often have need for specialized equipment, including medical drains and tubes. Specifically, neonatal intensive care units have equipment for stabilizing and monitoring severely ill newborns. Some equipment is used by almost every infant that requires intensive care. A non-limiting listing of examples of the equipment that is commonly used in the treatment or monitoring of pediatric patients includes the following:

(1) Cardiac monitoring device, including but not limited to adhesive patches with wires attached to the baby's chest, abdomen and/or leg monitor heart rate and rhythm;

(2) Arterial pressure monitor, such as when an umbilical artery catheter is placed in an artery in the umbilical cord stump that is left after the cord is cut in the delivery room for the measurement of arterial blood pressure;

(3) Catheters, including but not limited to an umbilical artery catheter, may be used for providing parenteral fluids or medications, or for removal of blood for testing, or a central line catheter may be threaded into the heart to measure central venous pressure;

(4) Nasogastric or orogastric tubes may be placed through the nose or mouth into the stomach and used to provide nutrition, or to remove air or gastric contents;

(5) Peripheral intravenous lines are attached generally through veins in the hand, arm, foot, lower leg or scalp. Pediatric patients can receive fluid, nutrition, and medication through these lines;

(6) Urinary catheter and drainage bags are used to facilitate urine elimination. Urine from the catheter can be collected in the drainage bag, from which it can be removed and measured. The catheter generally is placed through the urethral opening of the patient into the bladder. Urine flows through the tube into a drainage bag for measurement. The urine may be tested to evaluate the baby's well-being;

(7) Chest tubes may be used in instances in which the pediatric patient has pathology such as a collapsed lung, in which case a chest tube may be placed in the area the lung should occupy. The tube may be connected to a drainage system which creates a suction that allows the collapsed lung to re-expand into the space and heal. Drainage is removed by the chest tube and collects in the drainage system;

(8) Nasal canulas may be used to deliver oxygen through the nose, or positive airway pressure systems, which will include an endotracheal tube and may require mechanical ventilation may be used;

(9) Abdominal drains may be placed for the management of conditions such as peritonitis. An example of such a drain is a Jackson-Pratt tube;

(10) Nephrostomy tubes may be needed, and are placed percutaneously into the abdominal area to the kidneys. These tubes facilitate the collection of urine directly from the kidney to the outside of the body;

(11) Gastrostomy tubes may be required for pediatric patients with medical problems that prevent nutrition by mouth. A gastrostomy tube is inserted percutaneously through the abdomen directly into the stomach to allow the delivery of nutrition directly to the stomach.

Other tubes and devices that are routinely used in pediatric patients include dialysis tubes, insulin pumps, and analgesia tubes.

When medical tubes or drains are used in pediatric patients, it is important to secure the tubes or drains in the proper location. Especially regarding tubes placed percutaneously into the abdomen, it is essential to protect the insertion site, avoid dislodgement of the tube or drain, and to avoid kinking or obstruction of tube or drain. This is necessary to prevent any event that would interrupt the intended outcome. Accordingly, properly securing the tube/device is necessary to support a patient's recovery and medical management.

Presently available methods or devices for securing tubes or drains or other devices used in pediatric patients are inadequate. A commonly used method is using tape or other adhesives to secure the tubes or drains to the pediatric patient. But this method has proven to be difficult to use successfully. Proper taping is necessary, and in cases where the parents or another caregiver outside of the hospital setting is required to do the taping, it is often performed inadequately. Even at its best, adhesives result in unnecessary trauma to the pediatric patient.

Various tube or drain management garments or devices have been described in the literature. U.S. Application 2005/0020995 discloses a peritoneal dialysis security band and methods of use. The invention in that application is a security band having an elastic band with a catheter retaining pocket positioned along a portion of the elastic band. U.S. Pat. No. 6,436,074 discloses a garment for securing and exposing a peritoneal dialysis catheter and catheter exit site. This invention enables replaceable gauze to be held in place at a peritoneal catheter site without using tape, doing so by using a torso belt with a block of foam rubber disposed therein, with the end of the catheter secured in an external pouch. U.S. Pat. No. 5,468,229 discloses a peritoneal dialysis catheter support belt which is a belt having an aperture for receiving and orienting the protruding portion of an implanted catheter. U.S. Pat. No. 5,853,396 discloses a tuck-away belt for peritoneal dialysis patients, which consists of a belt which allows the patient to enclose a protruding tube, with the belt comprising elastic band. A substantially similar dialysis catheter support undergarment is disclosed in U.S. Pat. No. 6,126,639. U.S. Pat. No. 7,854,020 discloses a safety drain holding system which has a medical support garment comprising a medical container holder having first and second ends and at least one pocket. The pocket is adapted to support at least one container for storing bodily fluid output from a patient to the container. The medical support garment comprises a belt including first and second ends adapted for releasable attachment to the first and second ends, respectively, of the medical container holder such that the pocket is held adjacent to the waist of the patient when the first and second ends of the belt are attached to the first and second ends, respectively, of the medical container holder. U.S. Pat. No. 5,244,464 discloses a band for securing and aligning medical tubing relative to a portion of a human body. The disclosed invention includes primary and secondary straps constructed of VELFOAM fastened to the foam substrate of the VELFOAM strap, which can be adjustably fastened to itself about a portion of a human body. The secondary strap includes one or more male VELCRO attached to the foam substrate of the VELFOAM attaches at its ends anywhere along the primary strap, trapping the medical tubing between its ends and the primary strap to secure the medical tubing in place. After review of presently available devices and the prior art, it is clear that none disclose a device or method suitable for use in pediatric patients to secure medical tubes or drains, or other devices, in place in the manner of the present invention,

### BRIEF SUMMARY OF THE INVENTION

The present invention is a pediatric abdominal drain or tube management device. This invention will protect, anchor and support any and all drains or devices inserted into the abdomen. The invention provides a non-adhesive non-invasive manner of anchoring abdominal drains. It includes a flexible light weight binder that covers the abdomen connected by a fastening means. The fastening means is preferably a hook and loop material, such as Velcro. There is another single piece of flexible material that covers the tube as well as a small piece of material connected to the binder that anchors the tube directly.

The disclosed invention anchors the abdominal drain/device in a non-invasive non-adhesive manner. This will not only protect, anchor and support the tube but give parents piece of mind. Traditionally tubes/devices are anchored with sutures and adhesive materials which quite often are not enough to prevent dislodgement of abdominal device. This invention will reduce the amount of adhesives needed. Another specific issue with managing tubes in children is dislodgement. For example, if a nephrostomy tube in an infant or child becomes dislodged, the child may require a hospital visit, and anesthesia and intubation to have the tube re-inserted. There is a concern to direct injury to the kidney such as hydronephrosis when the tube is not correctly placed, and there are always concerns related to the possible danger of repeated anesthesia and the repeated dangers of the procedure of inserting a new nephrostomy tube. The present invention will help prevent an infant or child from being able to pull on the tube. The invention completely covers the tube/device helping to prevent the child from dislodging the tube. This is particularly helpful when the parents and child are sleeping. The cover will allow the child full range of motion and give parents piece of mind, as well as allowing easy access to the insertion site. Further, the present invention can be placed over clothing to decrease any irritation to skin. It is an inexpensive, noninvasive and reusable way to manage abdominal drains and devices.

An embodiment of the present invention is a device that can be fabricated by beginning with a one piece flexible abdominal band, similar to an abdominal binder. The abdominal band wraps around the back of the patient and connects in the front, preferably with a hook and loop material, such as Velcro. Another piece of flexible material is connected directly to the first piece on the flank, preferably with Velcro on one end. This second piece will cover the tube as it exits the initial abdominal band. The abdominal band piece comprises slits on either side of the binder on the anterior side of the abdominal band where the tube exits the body. The slits preferably comprise Velcro as well. There are preferably two Velcro connectors that encircle the tube directly. There is one connector on the right and one on the left. The one that will be used depends upon what side of the body the tube is on if there is only one tube. Both connectors may be used in patients having bi-lateral tubes. The drainage collection bag is connected directly to the binder and can hang there to give the parent freedom when carrying their child.

This summary of the invention does not necessarily describe all features of the invention.

### BRIEF DESCRIPTION OF THE SEVERAL VIEWS OF THE DRAWINGS

These and other features of the invention will become more apparent from the following description in which reference is made to the appended drawings wherein:

FIG. 1 is a plane view of an embodiment of the present invention.

FIG. 2 is a plane view of the present invention with a patient ready for use of the device.

FIG. 3 is a plane view of an embodiment of the invention, being initially applied to the patient.

FIG. 4 is a plane view of an embodiment of the present invention, showing further application to the patient.

FIG. 5 is a plane view of an embodiment of the present invention fully applied to the patient, but with tube and bag uncovered.

FIG. 6 is a plane view of an embodiment of the present invention fully applied to the patient with the tube and bag covered.

### REFERENCE NUMERALS IN THE DRAWINGS

### Detailed Description of Various Embodiments of the Invention

Various embodiments of the invention are described more fully hereinafter with reference to the accompanying drawings, in which some, but not all embodiments of the invention are shown in the figures. Indeed, these inventions may be embodied in many different forms and should not be construed as limited to the embodiments set forth herein; rather, these embodiments are provided so that this disclosure will satisfy applicable legal requirements.

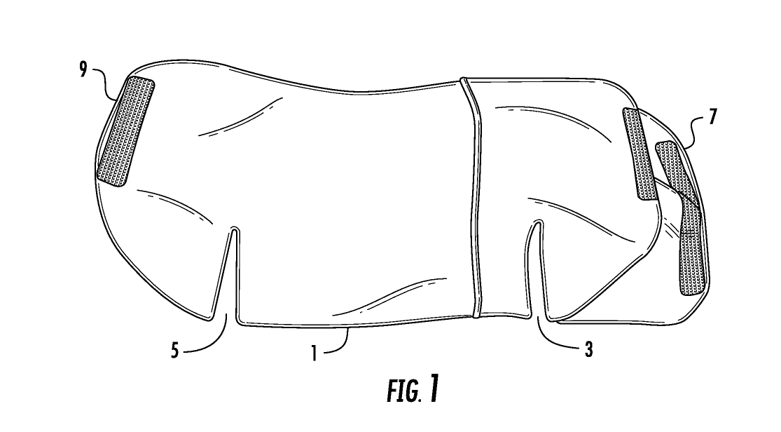

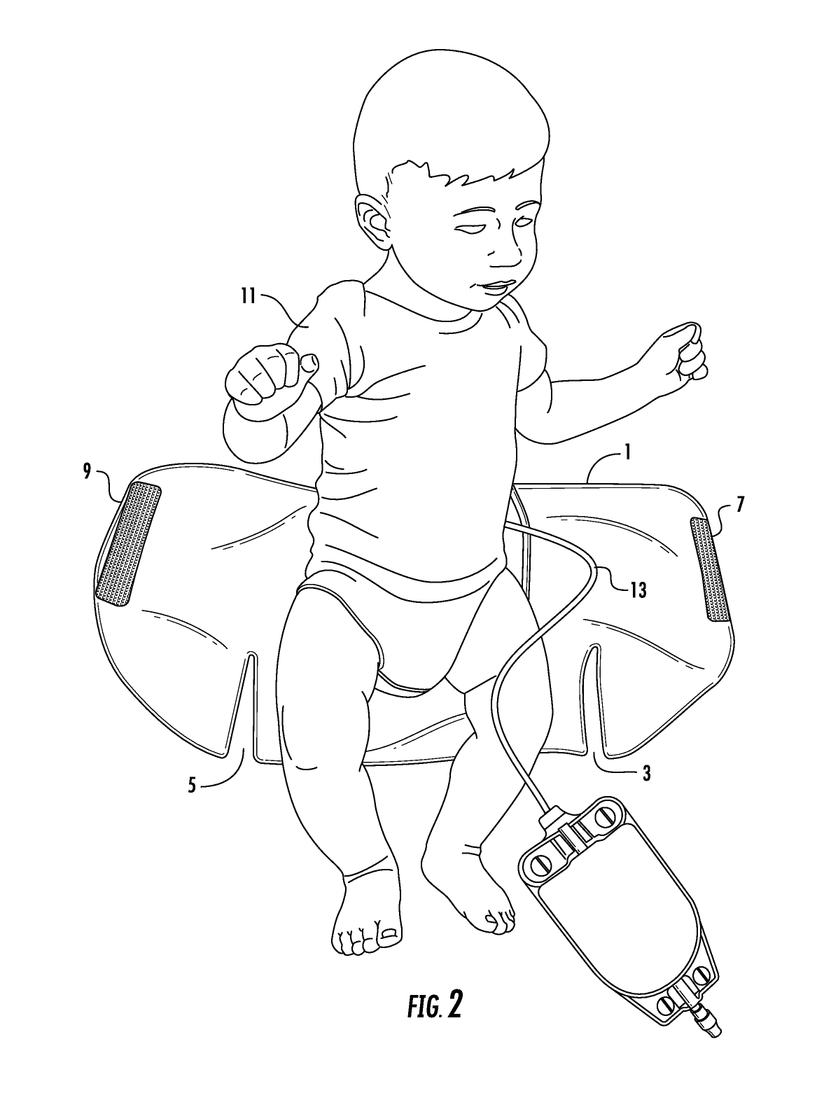

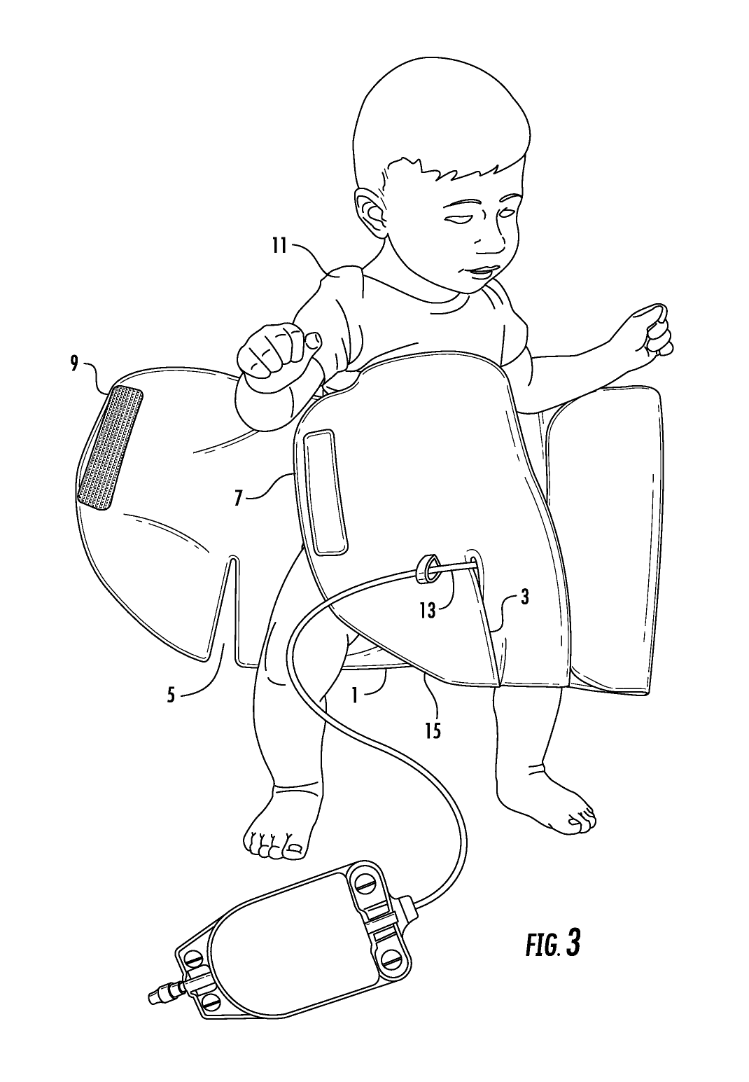

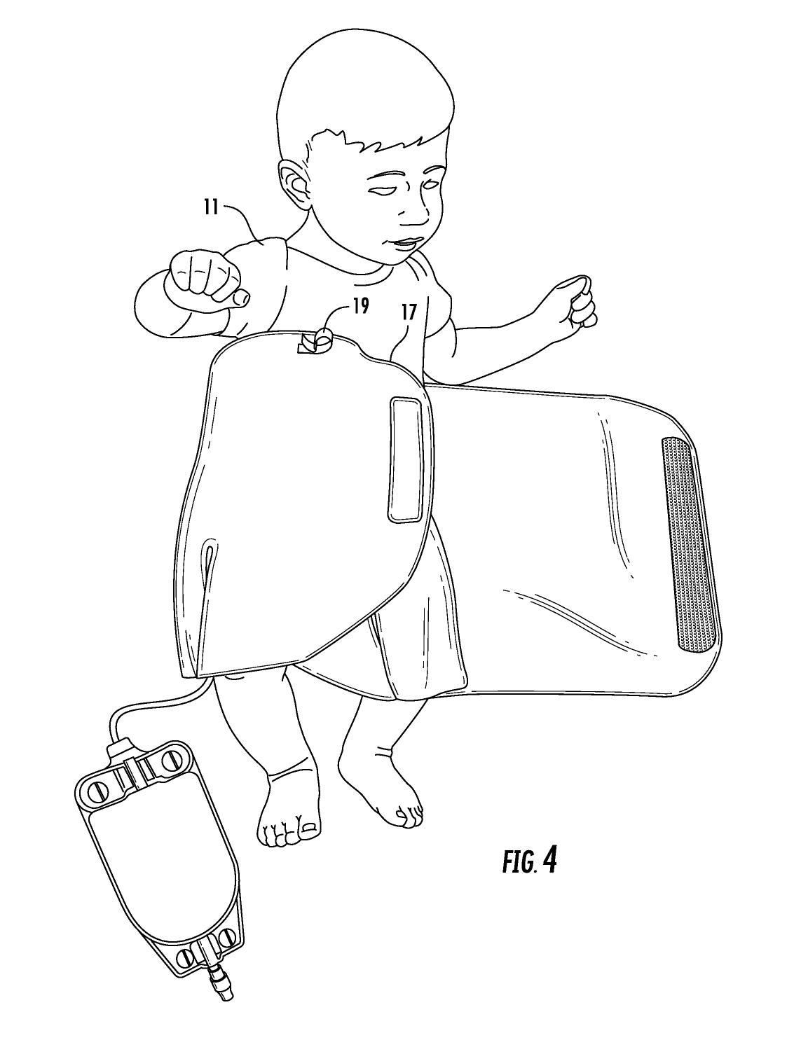

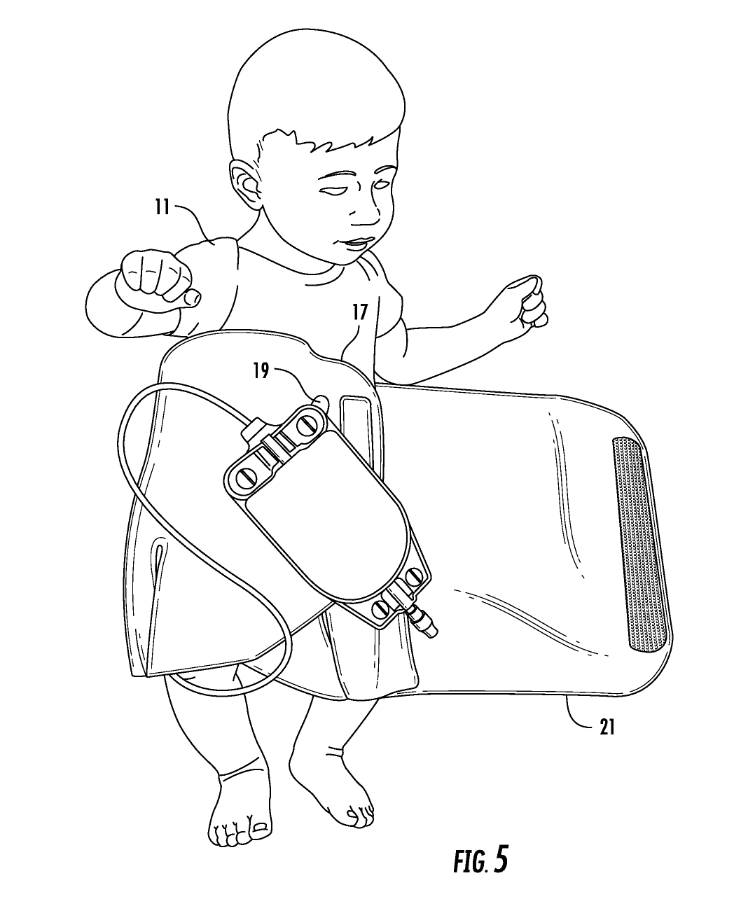

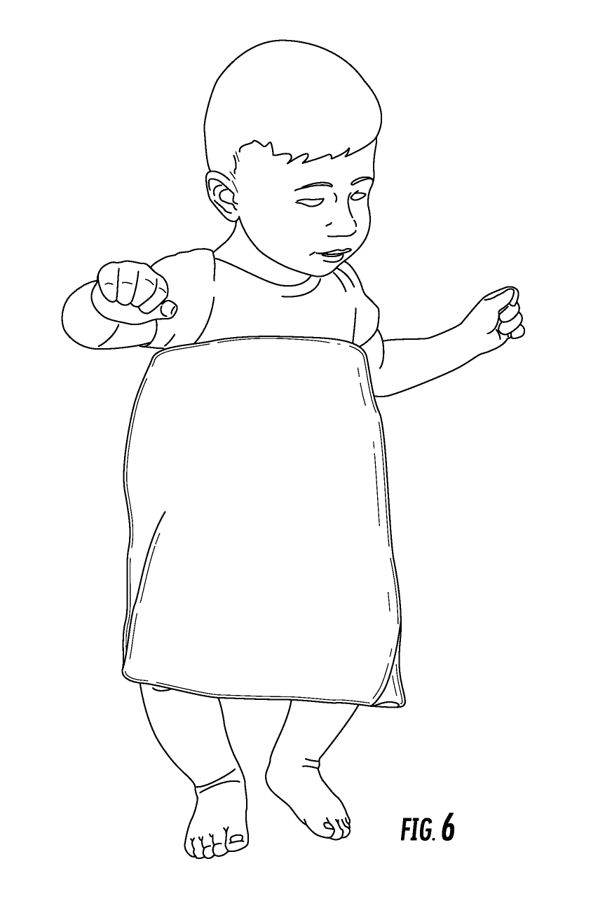

A first embodiment of the present invention is shown in detail in FIGS. 1-6. FIG. 1 shows the present invention. In a non-limiting example, the main portion 1 where the pediatric patient is initially placed is approximately 11 inches wide and 30 inches long. In a non-limiting example, there is a first lateral slit 3 and second lateral slit 5, approximately 5 inches long, located approximately 7 inches from the first lateral edge 7 and second lateral edge 9 of the device. FIG. 2 shows the first step in applying an embodiment of the present invention to a pediatric patient, with the pediatric patient 11 being placed on the device. In FIG. 2, a drain 13 extending from the pediatric patient 11 is on the left side. FIG. 3 shows a further step in applying an embodiment of the present invention to a pediatric patient, with the first lateral flap portion 15 on the same side of the drain folded over the patient 11, with the drain 13 or tube placed through the first lateral slit 3 and being held in place by Velcro. FIG. 4 shows a further step in applying an embodiment of the present invention, with the second lateral flap portion 17 folded over the first lateral flap with Velcro fastener 19 at the top for securing the drain 13. FIG. 5 shows the second lateral flap portion 17 on the opposite side of the drain folded over and the drain bag hooked to the Velcro fastener 19 on top of the outside of the second flap portion 17. FIG. 6 shows the covering portion 21 covering the drain bag.

In a preferred embodiment, the present invention is a pediatric drain or tube management device. The device comprises a unitary swaddling device having a main portion, a first lateral portion adjacent to the main portion, a second lateral portion adjacent to the main portion and opposite the first lateral portion, and a covering portion. Each lateral portion has an aperture extending therethrough and an end having a fastening means affixed thereto. The covering portion is positioned exterior to the lateral portions and has a fastening means affixed thereto. By swaddling device, applicant simply means a device that can be applied in a manner that wraps the pediatric patient. In a further embodiment, the present unitary swaddling device is configured such that a pediatric patient having a medical tube is placed upon the main portion of the device, the first lateral portion is folded over the patient and the second lateral portion is folded over the first lateral portion, such that the medical tube is extended through the apertures in the lateral portions and the fastening means is used to secure the drain in place, and the covering portion is folded over the lateral portions and the secured drain. In a further embodiment, the pediatric tube or drain management device in which the fastening mean is any of one or more of the group consisting of hook and loop fastener, buttons, and zippers.

A further embodiment of the present invention is a method of management of tubes or drains in a pediatric patient having a medical tube. The method comprises placing a pediatric patient having a medical tube upon a swaddling device in a position such that the medical tube is substantially centered upon the swaddling device, with the swaddling device having two lateral portions with apertures therethrough. The method further includes a step of folding the first lateral portion of said swaddling device over the patient and arranging the tube through the aperture in that first lateral portion. The method further includes the step of folding the second lateral portion over the first lateral portion and arranging the tube through the aperture in the second lateral portion. The first and second lateral portions are fastened to secure the lateral portions and the tube in place, and the lateral portions and the secured drain are covered.

A further embodiment of the present invention is a method of management of tubes or drains in a pediatric patient having a medical tube. The method includes placing the pediatric patient having a medical tube upon a substantially center portion of a garment, folding a first lateral garment over the patient and arranging that first lateral portion garment in a manner holding the tube substantially stationary relative to the patient, folding a second lateral garment over the first lateral garment and arranging the second lateral garment in a manner holding the tube substantially stationary relative to the patient, and covering the lateral garments and the secured drain. In an embodiment, the center portion of the garment, said first lateral garment, said second lateral garment, and said cover are all provided in a single unitary garment device.

A further embodiment of the present invention comprises a kit having the presently disclosed devices.

The advantages of the present invention, without limitation, include the following:
Inexpensive—The present invention can be provided in a washable and reusable form. Current devices using adhesives are disposable. While the reusability of the device is an advantage of the washable and reusable forms, the present invention may also be provided in a disposable form.
Non-adhesive—This invention includes no adhesives thereby limiting damage to skin. Adhesives are soiled often and repeated changes to adhesives can cause considerable damage to the skin. This invention will decrease the amount of adhesives needed.
Simplicity—This device is a single device managed with fastening materials, preferably Velcro. It is made to be removed in a simple and safe manner.
Tube support—The tube is anchored by a simple two part holder that will encircle the tube and ensure that the tube does not kink/bend or is not dislodged from being pulled on or if a drainage bag attached to the tube is dropped.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed. Moreover, the terms “consisting”, “comprising” and other derivatives from the term “comprise” are intended to be open-ended terms that specify the presence of any stated features, elements, steps, or components, and are not intended to preclude the presence or addition of one or more other features, elements, integers, steps, components, or groups thereof. Moreover, Applicants have endeavored in the present specification and drawings to draw attention to certain features of the invention, it should be understood that the Applicant claims protection in respect to any patentable feature or combination of features referred to in the specification or drawings. The drawings are provided to illustrate features of the invention, but the claimed invention is expressly not limited to the illustrated embodiments.

I claim:

1. A pediatric drain or tube management device comprising a unitary swaddling device having a main portion, a first lateral portion adjacent to the main portion, a second lateral portion adjacent to the main portion and opposite the first lateral portion, and a covering portion,
wherein each lateral portion has an aperture extending therethrough and an end having a fastening means affixed thereto, and
wherein said covering portion is positioned exterior to the lateral portions and has a fastening means affixed thereto.

2. The pediatric tube or drain management device of claim 1, wherein said unitary swaddling device is configured such that
(1) a pediatric patient having a medical tube is placed upon the main portion of the device,
(2) the first lateral portion is folded over the patient and the second lateral portion is folded over the first lateral portion, such that the medical tube is extended through the apertures in the lateral portions and the fastening means is used to secure the drain in place, and
(3) the covering portion is folded over the lateral portions and the secured drain.

3. The pediatric tube or drain management device of claim 1, wherein said unitary fastening means is selected from the group consisting of hook and loop fastener, buttons, and zippers.

4. A method of management of tubes or drains in a pediatric patient having a medical tube, said method comprising:
(1) placing said pediatric patient having a medical tube upon a swaddling device in a position such that said medical tube is substantially centered upon the swaddling device, said swaddling device having two lateral portions with apertures therethrough,
(2) folding the first lateral portion of said swaddling device over the patient and arranging said tube through said aperture in said first lateral portion,
(3) folding the second lateral portion over the first lateral portion and arranging said tube through said aperture in said second lateral portion,
(4) fastening said first and second lateral portions to secure the lateral portions and the tube in place, and
(5) covering the lateral portions and the secured drain.

5. A method of management of tubes or drains in a pediatric patient having a medical tube, said method comprising:
(1) placing said pediatric patient having a medical tube upon a substantially center portion of a garment,
(2) folding a first lateral garment over the patient and arranging said first lateral portion garment in a manner holding said tube substantially stationary relative to said patient,
(3) folding a second lateral garment over the first lateral garment and arranging said second lateral garment in a manner holding said tube substantially stationary relative to said patient, and
(4) covering the lateral garments and the secured drain.

6. The method of claim 5 wherein said center portion of a garment, said first lateral garment, said second lateral garment, and said cover are all provided in a single unitary garment device.

7. A kit comprising the device of claim 1.

8. A kit comprising the unitary garment device of claim 6.
